(19) United States
(12) Patent Application Publication
(10) Pub. No.: US 2006/0246571 A1
(43) Pub. Date: Nov. 2, 2006

(54) Apparatus and method for automatically producing substance-introduced particles

(75) Inventors: Sachihiro Youoku, Kawasaki (JP); Jun Sasaki, Kawasaki (JP); Junichi Murakoso, Kawasaki (JP); Nobuhiko Onda, Kawasaki (JP); Kazuo Tamamushi, Kawasaki (JP); Kiyoshi Taninaka, Kawasaki (JP)

Correspondence Address: STAAS & HALSEY LLP, SUITE 700, 1201 NEW YORK AVENUE, N.W., WASHINGTON, DC, 20005, US

(73) Assignee: FUJITSU LIMITED, Kawasaki (JP)

(21) Appl. No.: 11/117,771

(22) Filed: Apr. 29, 2005

(51) Int. Cl. C12N 15/00

(52) U.S. Cl. 435/285.1; 435/286.2; 435/455

(57) ABSTRACT

Proposed is an apparatus for automatically producing particles having introduced therein a substance. The apparatus includes a feeding device that feeds a suspension containing a plurality of particles; a holding device that receives the suspension from the feeding device and immobilizes and release at least one particle reversibly; an injecting device that injects a substance into the particle while the particle has been immobilized by the holding device; and a removing device that removes particles that are not immobilized by the holding device.

### BACKGROUND OF THE INVENTION

1) Field of the Invention

The present invention relates to technology for injecting a substance into particles that are suspended in a suspension.

2) Description of the Related Art

Cells in which genes or drugs or both have been introduced are increasingly used in the fields of regeneration therapy and genome-based drug discovery. As a result, various methods to introduce various substances into cells are being researched. However, the methods that can be used for the medical purpose are different from those for the research purpose.

The methods that can be used for the medical purpose need to fulfill following three conditions:

a) Can handle variety of cells and substances;

b) Can introduce the substance into the cells efficiently; and

c) Can produce the substances introduced cell in large amounts.

In particular, the condition c) is important; because, in the regeneration therapy, it is said that 10⁵ to 10⁶ cells are necessary at a time.

Examples of known methods for introducing the substances into the cells include:

(1) Biological techniques, such as a vector method;

(2) Chemical techniques, such as a transfection method; and

(3) Physical techniques, such as electroporation method, particle gun method, and injection method.

Among these, the biological techniques and the chemical techniques have been widely used in molecular biological studies. However, these techniques use viruses or bacteria and depend on specific combination of the kind of cells with the kind of the substance to be introduced. Accordingly, the biological techniques and the chemical techniques are not suitable for use in regeneration therapy although they can be used in research.

Among the physical techniques, the electroporation method includes breaking the cell membrane to form an opening to inject a gene in the cell (see, for example, Japanese Patent Application Laid-Open Publication No. H11-018770 and Japanese Patent Application Laid-Open Publication No. H11-506630); moreover, the particle gun method includes accelerating a minute cell to which a gene is attached so that the minute cell hits a bigger cell to break the cell membrane of the bigger cell to form an opening through which the minute cell is introduced into the cell to convey the gene (see, for example, Japanese Patent Application Laid-Open Publication No. H06-062871 and Japanese Patent Application Laid-Open Publication No. H09-248183). These methods do not depend on the specific combination of the cell with the substance to be introduced; however, these methods have a problem that it is difficult to control the device used so that the rate of successful introduction is very low.

Injection methods have been disclosed in, for example, Japanese Patent Application Laid-Open Publication No. H05-192171, Japanese Patent Application Laid-Open Publication No. H06-343478, Japanese Patent Application Laid-Open Publication No. 2000-023657, Japanese Patent Application Laid-Open Publication No. 2002-027969, and Japanese Patent Application Laid-Open Publication No. H01-112976. The injection methods are highly successful in introducing the substances and do not depend on specific combinations of the cell with the substance to be introduced. Therefore, the injection methods are conceived most reliable.

However, the injection methods have a problem. The injection methods involve manual operations on a Petri dish under a microscope and the operator must be skilled. Even highly skilled operators can handle only a few hundreds of cells per hour. Thus, the throughput of the injection methods is very low. Moreover, after the substance is introduced into cells, the operator must manually transfer the cells to a cell cultivating device. This may increase the possibility of contamination.

Some of the injection methods involve arranging cells in a one-dimensional or a two-dimensional array before performing injection. Such methods are disclosed in, for example, Japanese Patent Application Laid-Open Publication No. 2000-023657, Japanese Patent Application Laid-Open Publication No. 2002-027969, and Japanese Patent Application Laid-Open Publication No. H01-112976. However, these methods have a problem that feeding and taking out cells are not taken into consideration. Therefore, these methods have low throughputs and cannot be used directly in the field of regeneration therapy and in the field of an industry such as genome-based drug discovery.

Moreover, in artificial insemination, egg cells are handled individually, that is cell by cell. On the contrary, in the case of gene introduction targeted at general cells, cells are handled as a group. In the conventional injection methods, cells are arranged at random on a Petri dish, so that the cells after the substance has been introduced must be handled as a group.

As described above, it is convenient to handle the cells as a group when the number of cells is large. However, this is not suitable for some applications, for example, observation of a single or only a small number of cells to see if the cell or cells exhibit an effect of interest as is expected for medical purposes.

Moreover, currently, there are no apparatus and method for automatically producing substance-introduced particles that automatically perform such a series of steps and no means for producing substance-introduced particles in large amounts is available.

### SUMMARY OF THE INVENTION

It is an object of the present invention to solve at least the problems in the conventional technology.

According to an aspect of the present invention, an apparatus for producing particles having introduced therein a substance includes a feeding device that feeds a suspension containing a plurality of particles; a holding device that receives the suspension from the feeding device, immobilizes at least one particles while the substance is being introduced in the particles and releases the particle once the substance is introduced in the particles; an injecting device that injects the substance into the particles while the particles are immobilized by the holding device; and a removing device that removes particles that are not immobilized by the holding device.

According to another aspect of the present invention, a method of producing particles having introduced therein a substance includes feeding a suspension containing a plurality of particles; immobilizing at least one of the particles; injecting the substance into the particle that has been immobilized; releasing the particle that has been immobilized and into which the substance has been injected; and removing particles that are not immobilized at the immobilizing.

The other objects, features, and advantages of the present invention are specifically set forth in or will become apparent from the following detailed description of the invention when read in conjunction with the accompanying drawings.

### BRIEF DESCRIPTION OF THE DRAWINGS

FIG. 1 is a schematic cross section for explaining the principle of the present invention;

FIG. 2 is a schematic for explaining an apparatus for automatically producing substance-introduced particles according to a first embodiment of the present invention;

FIG. 3 is a perspective of a micro vessel shown in FIG. 2;

FIG. 4 is schematic for explaining an example of how the cell is fixed in the micro vessel shown in FIG. 3;

FIG. 5 is a flowchart of the operations performed by each of the devices shown in FIG. 2;

FIG. 6 is a continuation of the flowchart shown in FIG. 5;

FIG. 7 is a schematic for explaining a mechanism of temporary holding and releasing cells according to the first embodiment of the present invention;

FIG. 8 is a schematic for explaining how the cells are fixed in a micro vessel of an apparatus for automatically producing substance-introduced particles according to a second embodiment of the present invention;

FIG. 9 is a perspective of a micro vessel of an apparatus for automatically producing substance-introduced particles according to a third embodiment of the present invention; and

FIG. 10 is a schematic plan view of a transporting device according to a fourth embodiment of the present invention.

### DETAILED DESCRIPTION

Exemplary embodiments of the present invention are explained in detail with reference to the accompanying drawings.

As used herein the term “particle having introduced therein a substance” is referred to as “substance-introduced particle”, and the term “apparatus for automatically producing substance-introduced particles” is also referred to as “automatic substance-introduced particles producing apparatus” or simply as “automatic production apparatus”. Similarly, the term “method for automatically producing substance-introduced particles” is also referred to as “automatic substance-introduced particles producing method” or simply as “automatic production method”.

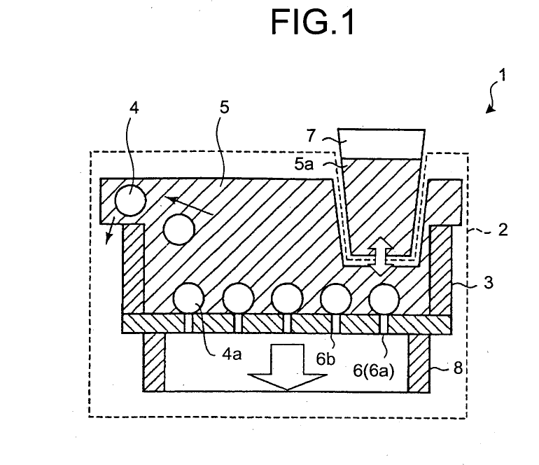

FIG. 1 is a schematic for explaining the principle of the present invention. As shown in FIG. 1, an automatic substance-introduced particles producing apparatus 1 according to an embodiment of the present invention, that produces substance-introduced particles, for example, cells, includes a particle holding device 2. The particle holding device 2 has a micro vessel 3. The micro vessel 3 contains a suspension 5 fed from a feeder (not shown). Particles 4 are suspended in the suspension 5. A substance is introduced into the particles 4. The micro vessel 3 is provided with particle immobilizing units 6a, 6b, . . . , (collectively referred to as “particle immobilizing unit 6”), for example, on the bottom thereof. The particle immobilizing unit 6 temporarily holds the particles 4 in such a manner that the particle does not move about. Thus, the particle immobilizing unit 6 temporarily immobilizes the particles at predetermined positions. The automatic substance-introduced particles producing apparatus 1 further includes a particle removing device 7. The particle removing device 7 is used to remove those particles that are not immobilized in the particle holding device 2.

The particle holding device 2 can be provided with the single micro vessel 3 or a plurality of the micro vessels 3. Alternatively, a plurality of the micro vessels 3 can be arranged on a plate to constitute a single particle holding device. The micro vessel 3 can be made of a transparent material, for example an organic transparent material such as polycarbonate or acrylic resin, or an inorganic transparent material such as glass. When the micro vessel 3 is made of a transparent material, the particles can be observed with transmitted light and sharp images of the particles, for example, cells can be obtained.

The feeder can include a suspension feed controlling device that controls the amount of suspension to be fed, a delivery device that delivers the suspension 5, and a suspension agitating device that agitates the suspension 5. The delivery device can feed the suspension 5 through piping (not shown) to the particle holding device 2.

The particle immobilizing unit 6 can have various forms. The one shown in FIG. 1 is in the form of a plurality of openings 6a formed in, for example, the bottom of the micro vessel 3. In this case, a sucking device 8 that sucks only a liquid 5a in the suspension through the openings 6a is provided and connected to the openings 6a. When the sucking device 8 applies negative pressure, the suspension 5 is sucked and the particles 4 suspending in the suspension 5 are held, or trapped, at the openings 6a. When the sucking device 8 applies positive pressure, the particles are released from the openings 6a. In this manner, the particles can be temporarily immobilized and then released.

Alternatively, the particle immobilizing unit 6 can be realized by coating, for example, the bottom of the micro vessel 3 with a particle adhering substance (not shown) whose adhesion to the particle can be varied. For example, the hydrophilicity/hydrophobicity of the particle adhering substance can be switched by changing the temperature of the particle adhering substance to temporarily immobilize the particles and then release.

The particle removing device 7 can include a liquid feed controlling device (not shown) that controls the amount of the liquid 5a to be delivered, a delivery device (not shown) that delivers the liquid 5a. The particle removing device 7 can be connected to the particle holding device 2 through piping (not shown). The particle removing device 7 feeds the liquid 5a that contains no particle to the particle holding device 2 through the piping to discharge the suspension 5 that contains the particles that are not immobilized out of the micro vessel 3.

Alternatively, the particle removing device 7 can include a sucking device (not shown) and be connected to the particle holding device 2 through piping (not shown). In this case, the particle removing device 7 absorbs the suspension 5 that contains free particles, that is, particles that are not immobilized, from the particle holding device 2 through the piping and collects the free particles by, for example, filtering the suspension using a filter (not shown), and discharges the collected particles.

While the particles 4 are immobilized by the particle immobilizing unit 6, the substance can be introduced into the particles, for example, cells, by an injection method, in particular, microinjection method known in the art.

The automatic substance-introduced particles producing apparatus 1 can include a transporting device (not shown) that transports the particles after the substance is introduced therein and a storing device (not shown) that stores therein and manages the substance-introduced particles 4.

The transporting device can include a conveyor (not shown), which transports the micro vessel 3, and a drive controlling device (not shown), which controls the driving of the conveyor. The transporting device can transport the micro vessel 3 with the conveyor to the storing device.

Alternatively, the transporting device can include an operation arm (not shown) and a controlling device (not shown) that controls the operation arm and can fetch the micro vessel 3 to the storing device.

A typical example of the particle is a cell. However, the present invention is not limited to cells.

According to the present invention, a large amount of particles, e.g., cells, can be treated by an injection method that is highly reliable, that does not depend on specific combinations of particles and substances, and that enables treatment of the particles individually. The automatic substance-incorporated particles producing apparatus of the present invention can be applied to a gene-introducing apparatus for medical purposes, such as regeneration therapy and genome-based drug discovery.

The automatic substance-introduced particles producing method of the present invention is explained referring to FIG. 1. The method has six steps as described below:

Step 1: The particle holding device 2 is connected to the feeder through the piping and the suspension 5 with the particles 4 is fed into the micro vessel 3.

Step 2: The particles 4 in the suspension 5 are immobilized at the particle immobilizing unit 6 in the micro vessel 3.

Step 3: The particle removing apparatus 7 is connected to the particle holding device 2 through the piping and free particles 4 are removed.

Step 4: A substance is injected into the immobilized particles 4a, that is, particles that are held at the at the particle immobilizing unit 6, by an injecting device. The substance can be injected into the immobilized particles 4a by any know method.

Step 5: Once the substance is introduced into the immobilized particle 4a, the substance-introduced particle 4a, that is the immobilized particle 4a into which the substance has been introduced, is released.

Step 6: The micro vessel 3 is transported to the culturing device by the transporting device.

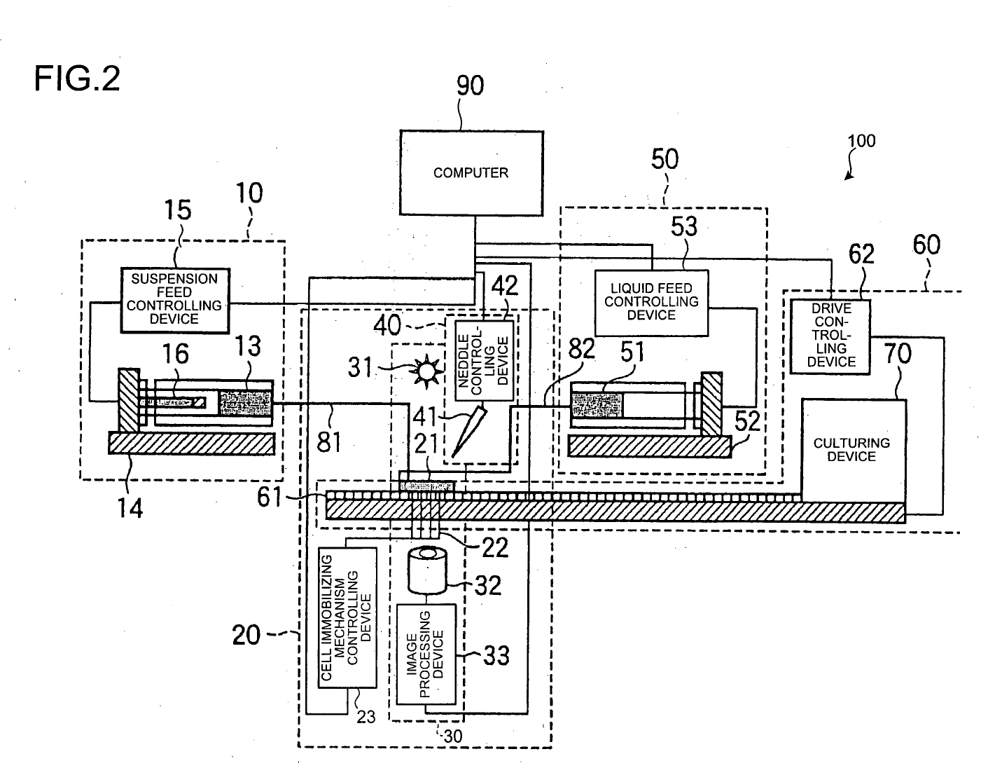

An automatic substance-introduced particles producing apparatus and an automatic substance-introduced particles producing method according to a first embodiment of the present invention are explained referring to FIGS. 2 to 7.

FIG. 2 is a schematic of the automatic substance-introduced particles producing apparatus 100 according to the first embodiment of the present invention. The automatic substance-introduced particles producing apparatus 100 includes a feeding device 10, a cell holding device 20, an injecting device 40, a removing device 50, a transporting device 60, and a culturing device 70.

The feeding device 10 includes a delivering device 14 that delivers a suspension 13 that includes a medium and cells suspended in the medium, a suspension feed controlling device 15 and a cell agitating device 16 provided in the delivering device 14. The suspension 13 in the delivering device 14 is agitated uniformly by the cell agitating device 16 and sent to a cell holding device 20 through piping 81.

The delivering device 14 is, for example, a syringe. The amount of the suspension delivered and the pressure of the suspension can be varied in response to instruction from the suspension feed controlling device 15. This configuration enables the amount of the suspension 13 delivered to the cell holding device 20 to be controlled.

The cell agitating device 16 includes a rotating rod that is used in the blood analysis and so on. However, any other agitating device can be used until it can uniformly agitate the cells in the suspension 13.

The cell holding device 20 includes a micro vessel 21 that contains the suspension 13 fed through the piping 81, a cell immobilizing unit 22 that is provided on the bottom of the micro vessel 21 and immobilizes the cells, and a cell immobilizing mechanism controlling device 23.

The cell holding device 20 further includes a cell monitoring device 30 that includes an image processing device 33 provided with a light source 31 and an objective lens 32. The cell monitoring device 30 always monitors the micro vessel 21 and detects completion of the immobilization by the cell immobilizing unit 22, and records information on the position of the immobilized cells.

The injecting device 40 includes a needle 41 for introducing a substance into the cells and a needle controlling device 42 that controls the position of the needle 41 by moving the needle 41. The injecting device 40 injects a gene or a drug solution into the cells immobilized at the cell immobilizing unit 22 using the needle 41.

The removing device 50 includes a delivering device 52 and a liquid feed controlling device 53. The delivering device 52 is, for example, a syringe that delivers a medium 51 therethrough. The medium 51 filled in the delivering device 52 is sent to the cell holding device 20 through piping 82 so that the cells that are not immobilized by the cell immobilizing unit 22 are removed by causing the suspension 13 containing free cells to overflow from the micro vessel 21. This makes it possible to immobilize in the micro vessel 21 only those cells into which a substance is introduced.

The transporting device 60 includes a conveyor 61 for transporting the micro vessel 21 and a drive controlling device 62 that controls the driving of the conveyor 61. The transporting device 60 transports the micro vessel 21 to a culturing device 70 that is a storing device for storing the cells after the introduction of the substance into the cells by the cell holding device 20 and the injecting device 40 is completed.

The culturing device can be a conventional one.

The automatic substance-introduced particles producing apparatus 100 includes a computer 90. The computer 90 controls the operations of the cell immobilizing mechanism controlling device 23, the cell monitoring device 30, the needle controlling device 42, and the suspension feed controlling device 15, the liquid feed controlling device 53, and the driving controlling device 62.

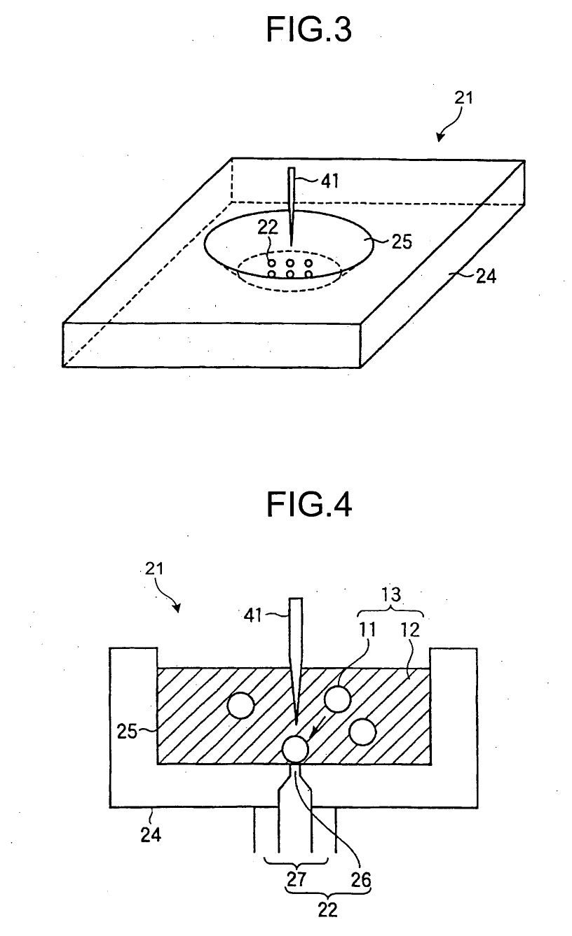

FIG. 3 is a perspective of an exemplary configuration of the micro vessel 21. The micro vessel 21 includes a plate made of transparent polycarbonate and a depression 25 having an inverse truncated cone shape. On the bottom of the depression 25 are provided a plurality of cell immobilizing units 22.

The inner volume of the depression 25 is preferably 300 microliters (μl) or less, for example, 150 μl. The cell immobilizing units 22 can be arranged at intervals of 25 micrometers (μm). A plurality of the cells can be immobilized in one micro vessel 21, so that the substance can be injected in a large number of cells at a time.

The micro vessel 21 is placed in the viewing field of a microscope that is provided in the image processing device 33. The cells in the micro vessel 21 are monitored with transmitted light and the states of the cells in the viewing field, such as the state of the cells that flow into the micro vessel 21, the sate of immobilization of the cells, immobilization positions of the cells, the state of the substance introduction, the states of the cell immobilizing units 22 and of the needle 41 are detected by the image processing device 33.

FIG. 4 is a schematic for explaining an example of how the cells are fixed in the cell immobilizing units 22. To make the illustration simpler, only one cell immobilizing unit 22 is depicted.

As shown in FIG. 4, the cell immobilizing unit 22 is constituted by an opening 26 provided on the bottom of a depression 25 in a plate 24 that constitutes the micro vessel 21. A cell immobilizing mechanism controlling device 23 is constituted by a sucking unit 27 that is closely attached to the opening 26.

Assuming that a cell 11 is a blood cell having a diameter of about 15 μm, the diameter of the depression 25 on the bottom thereof is preferably about 5 μm. The sucking unit 27 is made of polyether ether ketone (PEEK) that has excellent viscosity and has elasticity or it can be made of silicone resin. The sucking unit 27 is connected to a sucking means such as a syringe (not shown).

A medium 12 is sucked by the sucking means by an amount on the order of nanoliters (nl) to thereby trap and immobilize the cell 11 at the opening 26. The immobilized cell can be released by stopping the suction or by applying a positive pressure to the opening 26.

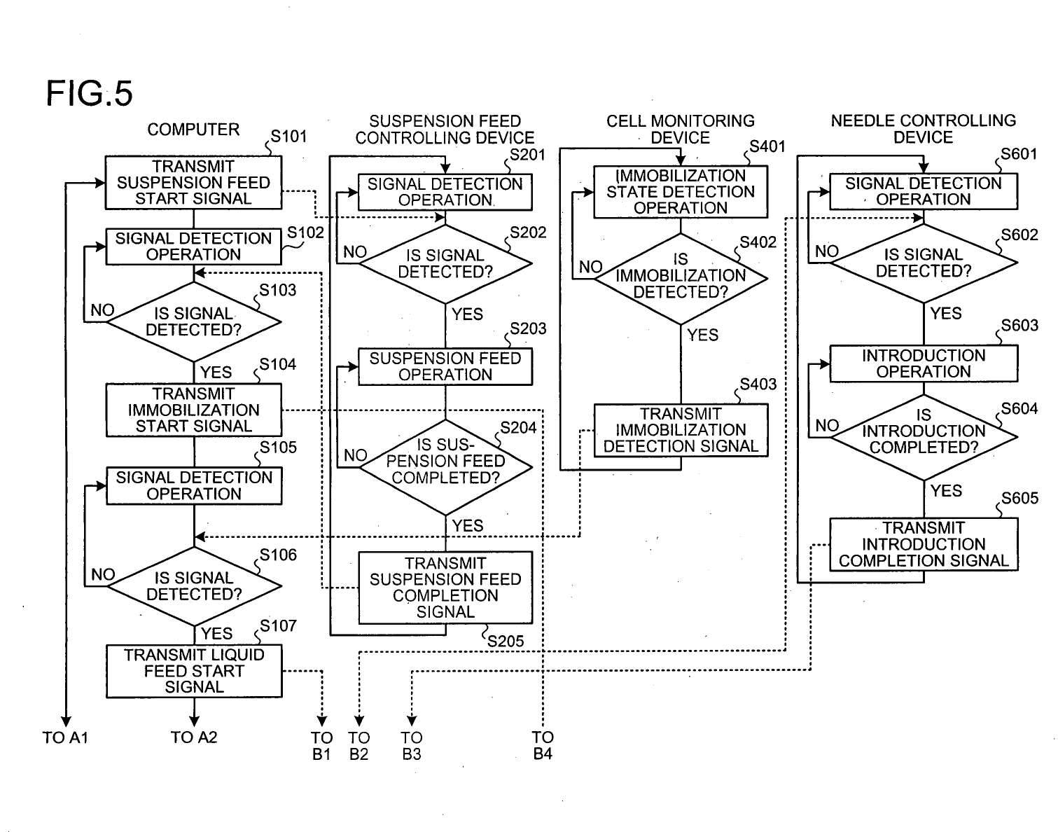

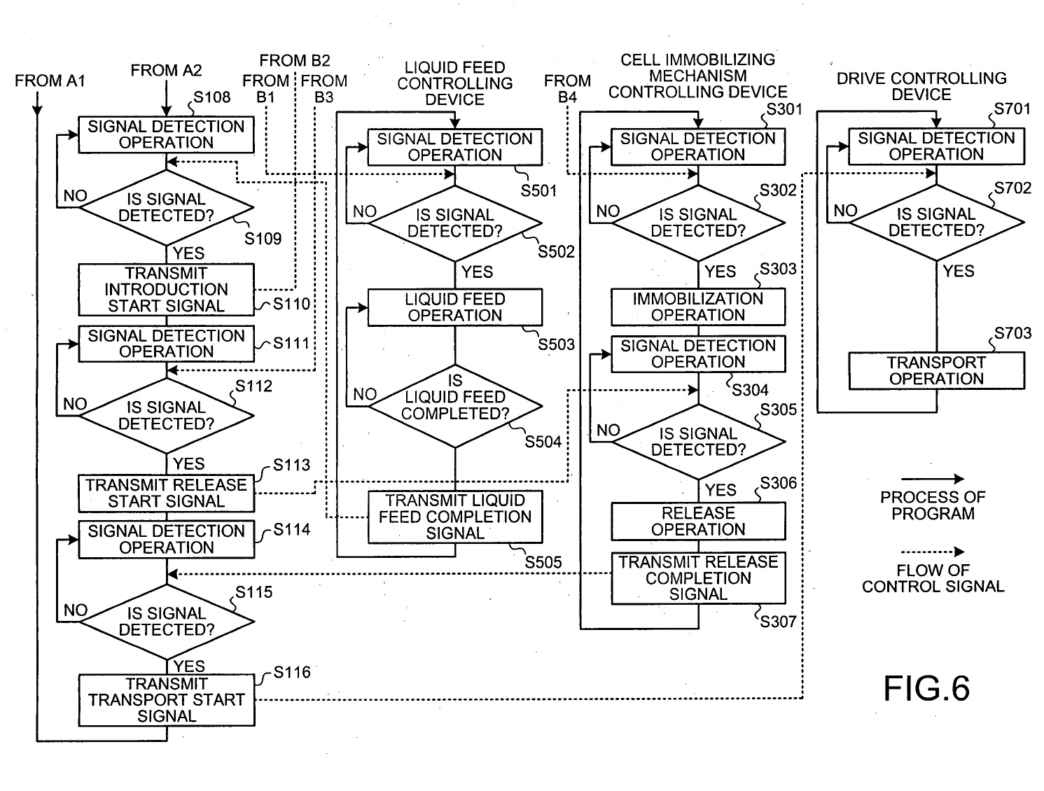

FIG. 5 is a flowchart of the operations performed by respective devices of the automatic substance-introduced particles producing apparatus 100. FIG. 6 is a continuation of the FIG. 5.

First, the suspension 13 is filled in the delivering device 14 and the suspension 13 is agitated uniformly in the delivering device 124 by the cell agitating device 16. When the cells 11 are sticky cells, the cells 11 are separated by treating them with trypsin.

The computer 90 transmits a suspension feed start signal (step S101). The suspension feed controlling device 15 detects whether the suspension feed start signal is received (step S201). When the suspension feed start signal is not detected (step S202, NO), the system control returns to step S201 and the suspension feed controlling device 15 continues the signal detection. When the suspension feed start signal is detected (step S202, YES), the suspension feed controlling device 15 controls the delivering device 14 to perform a suspension feed operation. That is, the uniformly agitated suspension 13 is sent to the cell holding device 20 through the piping 82 and is contained in the micro vessel 21 (step S203). After feeding of the suspension is started, when the suspension feed controlling device 15 does not detect completion of the suspension feed operation (step S204, NO), the suspension feed operation is continued. When the suspension feed controlling device 15 detects completion of the suspension feed operation (step S204, YES), the suspension feed controlling device 15 transmits a suspension feed completion signal to the computer 90 (step S205). The computer 90 detects whether the suspension feed completion signal is received (step S102). When the computer 90 does not detect the suspension feed completion signal (step S103, NO), the computer 90 continues the signal detection operation (step S102). When the computer 90 detects the suspension feed completion signal (step S103, YES), the computer 90 transmits an immobilization start signal to the cell immobilizing mechanism controlling device 23 (step S104).

The cell immobilizing mechanism controlling device 23 detects whether the immobilization start signal is received from the computer 90 (S301). When the immobilization start signal is not detected (step S302, NO), the cell immobilizing mechanism controlling device 23 continues the signal detection operation. When the immobilization start signal is received (step s302, YES), the cell immobilizing mechanism controlling device 23 controls the cell immobilizing unit 22 to perform immobilization operation (step S303). In this immobilization operation, the cells 11 are sucked through the openings 26 to trap and immobilize the cells 11 on the bottom of the depression 25 in the micro vessel 21.

The cell monitoring device 30 always monitors the inside of the micro vessel 21 and performs immobilization state detection operation, that is, checks to see whether the cells 11 are immobilized within the viewing field (step S401). When the immobilization state is not detected (step S402, NO), the cell monitoring device 30 continues the immobilization state detection operation. When the immobilization state is detected (step S402, YES), the cell monitoring device 30 transmits an immobilization state detection signal and information on positions of the immobilized cells to the computer 90 (step S403). The computer 90 detects whether the immobilization state detection signal is received from the cell monitoring device 30 (step S105). When the computer 90 does not detect the immobilization state detection signal (step S106, NO), the computer 90 continues the signal detection operation. When the computer 90 detects the immobilization state detection signal (step S106, YES), the computer 90 transmits a liquid feed start signal to the liquid feed controlling device 53 (step S107).

The liquid feed controlling device 53 detects whether the liquid feed start signal is received from the liquid feed controlling device 53 (step S501). When the liquid feed controlling device 53 does not detect the liquid feed start signal (step S502, NO), the liquid feed controlling device 53 continues the signal detection operation. When the liquid feed controlling device 53 detects the liquid feed start signal (step S502, YES), the liquid feed controlling device 53 controls the liquid feed device to perform a liquid feed operation (step S503). That is, the medium 51 is fed to the micro vessel 21 through the piping 82 to cause an overflow of the suspension in the micro vessel 21 to remove the free cells.

After the liquid feed is started, the liquid feed controlling device 53 monitors whether the liquid feed is completed. When the liquid feed is not completed (step S504, NO), the liquid feed controlling device 53 continues the liquid feed operation. When completion of the liquid feed is detected (step S504, YES), the liquid feed controlling device 53 transmits a liquid feed completion signal to the computer 90 (step S505).

The computer 90 detects whether the liquid feed completion signal is received from the liquid feed controlling device 53 (step S108). When the liquid feed completion signal is not detected (step S109, NO), the computer 90 continues the signal detection. When the liquid feed completion signal is detected (step S109, YES), the computer 90 transmits an introduction start signal (step S110).

The needle controlling device 42 detects whether the introduction start signal is received from the computer 90 (step S601). When the introduction start signal is not detected (step S602, NO), the needle controlling device 42 continues the signal detection operation. When the introduction start signal is detected (step S602, YES), the needle controlling device 42 performs an introduction operation (step S603). That is, the needle 41 is moved to the immobilization position at which the cells 11 are immobilized and the cell membrane of the cells 11 is stuck to make openings in the cell membrane through which openings the substance is injected into the cells 11.

The substance may be filled in the needle 41 or attached to the tip of the needle 41. When the substance is filled in the needle 41, a substance feed device (not shown) provided in the needle controlling device 42 is used to inject only a necessary amount of the substance into the cell 11.

The needle controlling device 42 monitors the introduction operation. When completion of introduction of the substance is not detected (step S604, NO), the needle controlling device 42 continues the introduction operation. When the completion of introduction is detected (step S604, YES), the needle controlling device 42 transmits an introduction completion signal to the computer 90 (step S605). The computer 90 detects whether the introduction completion signal is received from the needle controlling device 42 (step S111). When the introduction completion signal is not detected (step S112, NO), the computer 90 continues the signal detection operation. When the introduction completion signal is detected (step S112, YES), the computer 90 transmits a release start signal to the cell immobilizing mechanism controlling device 23 (step 113).

The cell immobilizing mechanism controlling device 23 detects whether the release start signal is received from the computer 90 (step S304). When the release start signal is not detected (step S305, NO), the cell immobilizing mechanism controlling device 23 continues the signal detection operation. When the release start signal is detected (step S305, YES), the cell immobilizing mechanism controlling device 23 controls the cell immobilizing units 22 to release the substance-introduced cells (step S306), and transmits a release completion signal to the computer 90 (step S307).

The computer 90 detects whether the release completion signal is received from the cell immobilizing mechanism controlling device 23 (step S114). When the release completion signal is not detected (step S115, NO), the computer 90 continues the signal detection operation. When the release completion signal is detected (step S115, YES), the computer 90 transmits a transport start signal to the drive controlling device 62 that controls the driving of the conveyor 61 (step S116).

The drive controlling device 62 detects whether the transport start signal is received from the computer 90 (step S701). When the transport start signal is not detected (step S702, NO), the drive controlling device 62 continues the signal detection operation. When the transport start signal is detected (step S702, YES), the drive controlling device 62 controls the conveyor 61 to perform a transport operation. That is, the conveyor 61 is operated to transport the micro vessel 21 to the culturing device 70 (step S703).

When the substance is injected into the cells 11, the substance-introduced cells are transported to the culturing device 70 where the substance-introduced cells are cultured and whether the effect of the substance is exhibited can be confirmed.

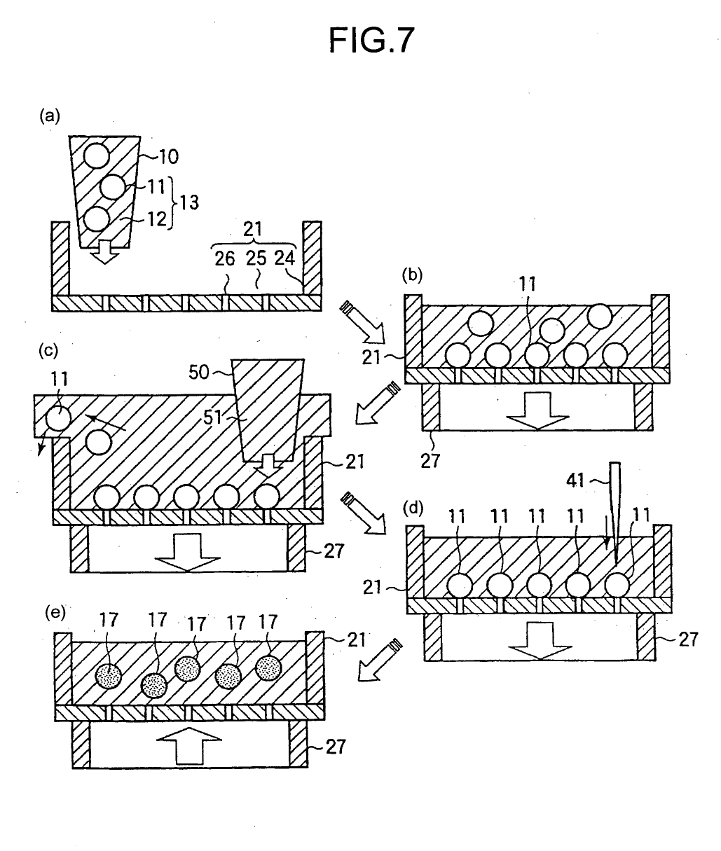

FIG. 7 is a schematic cross-section for explaining an example of the process including a series of operations from feed of the cells 11 to the micro vessel 21 to release of the substance-introduced cells according to the first embodiment of the present invention.

First, as shown in steps (a) to (e) in FIG. 7, the process from feed to release of the cells proceeds as follows:

Step (a): The suspension 13 is fed to the micro vessel 21 using the feeding device 10.

Step (b): The cell immobilizing mechanism controlling device 23 is attached to the side of the micro vessel 21 on which the openings 26 are provided to suck and immobilize the cell at the openings 26.

Step (c): Using the removing device 50, only the medium 51 is fed to the micro vessel 21 to remove the cells that are not immobilized.

Step (d): The needle 41 is operated to inject the substance into the cells 11.

Step (e): The cell immobilizing mechanism controlling device 23 is operated to stop the suction by the sucking unit 27 to release the substance-introduced cells 17 from the openings 26.

As explained above, according to the first embodiment of the present invention, the substance introduction operation can be automatically performed and a large amount of substance-introduced cells can be produced at a time.

In particular, since the removing device for removing cells that are not immobilized is provided, the adverse influence otherwise given by the non-immobilized cells that float randomly around the immobilized cells, such as disturbing the monitoring by the cell observation device, is obviated. Therefore, although an injection method is adopted, the substance injection operation can be performed efficiently, which increases the throughput.

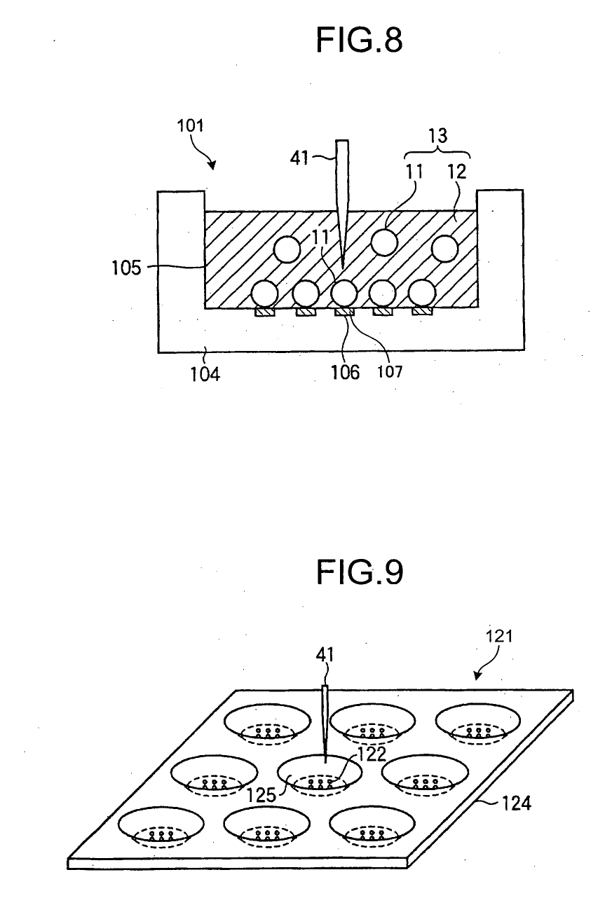

An automatic substance-introduced particles producing apparatus and an automatic substance-introduced particles producing method according to a second embodiment of the present invention are explained referring to FIG. 8. The basic configuration of the automatic substance-introduced particles producing apparatus is the same as that of the first embodiment and only the difference is in the micro vessel, so that only the micro vessel is explained.

FIG. 8 is a cross-section of a micro vessel 101 according to the second embodiment. The micro vessel 101 includes a plate 104 that is made of a transparent polycarbonate and provided with a depression 105 that has a rectangular cross-section. The bottom of the depression 105 is provided with a plurality of minute depressions 106 that has a rectangular cross-section. A cell adhering substance 107 is filled in each of the minute depressions. The inner volume of the depression 105 is preferably 300 μl or less, for example 150 μl.

In this case, examples of the cell adhering substance 107 include a lectin that bonds to a specified site of the cell 11 and a temperature-responsive polymer whose hydrophilicity and hydrophobicity can be controlled by the temperature. The cell 11 can be bonded to the lectin or the temperature-responsive polymer can be heated to make the polymer hydrophobic to bond and immobilize the cell 11 to the cell adhering substance 107.

The cells 11 are released by coating them with a proteolytic enzyme such as trypsin to separate the adhered surfaces of the cells. Alternatively, the temperature-responsive polymer can be cooled to convert the polymer hydrophilic and detach the cells 11 from the cell adhering substance 107.

In the second embodiment, since the cell adhering substance 107 is used as the cell immobilizing unit, the con figuration of the cell holding device 102 can be made simpler.

An automatic substance-introduced particles producing apparatus and an automatic substance-introduced particles producing method according to a third embodiment of the present invention are explained referring to FIG. 9. The basic configuration of the automatic substance-introduced particles producing apparatus is the same as that of the first embodiment and only the difference is in the micro vessel, so that only the micro vessel is explained.

FIG. 9 is a perspective of a micro vessel 121 according to the third embodiment. The micro vessel 121 includes a plate 124 that is made of a transparent polycarbonate and provided with a plurality of depressions 125 in the form of an inverse truncated cone. On the bottom of each of the depression 125 a plurality of cell immobilizing units 122 are coated. The inner volume of each of the depression 125 is preferably 300 μl or less, for example, 150 μl.

The number of the depressions 125 is not limited particularly and, for example, nine depressions 125 can be formed in the plate 124. Moreover, the configuration of the cell immobilizing unit 122 can be of a suction type in the same manner as that in the first embodiment or of a cell adhering substance type in the same manner as that in the second embodiment.

When there is a plurality of depressions, it is rather difficult to move the needle 41. One approach is to mount the micro vessel 121 on an x-y stage that can be moved as desired on a plane under the needle.

In the third embodiment, a large amount of cells can be processed at a time. Moreover, by using only one or only some of the depressions only a desired number of cells can be processed.

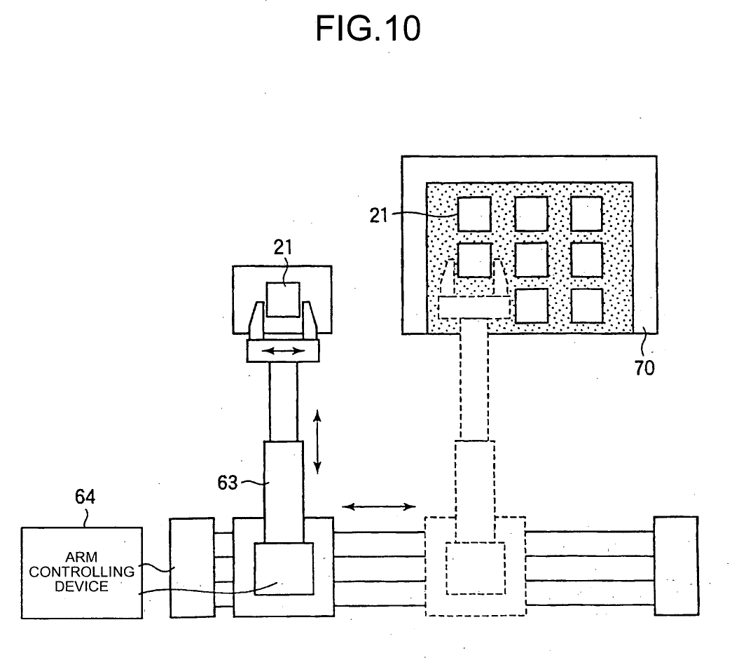

An automatic substance-introduced particles producing apparatus and an automatic substance-introduced particles producing method according to a fourth embodiment of the present invention are explained referring to FIG. 10. The basic configuration of the automatic substance-introduced particles producing apparatus is the same as that of the first embodiment and only the difference is in the transporting device, so that only the transporting device is explained.

FIG. 10 is a schematic plan view of a transporting device that constitutes a part of a cell holding device of the automatic substance-introduced particles producing apparatus according to the fourth embodiment. The transporting device includes a working arm 63 that is configured to hold and move a micro vessel 21 and an arm controlling device 64 that controls the working arm 63. After the substance is introduced into the cells, the arm controlling device 64 is operated to control the working arm 63 to grasp the micro vessel 21 and transport the micro vessel 21 to the culturing device 70. The micro vessel 21 can be the micro vessels explained in the second to the fourth embodiments.

The present invention is not limited to the conditions and configurations specifically described in the above-mentioned embodiments and various modifications and changes can be made. For example, numerical values of the inner volume, diameter, number of openings are not limited to those described and can be varied optionally depending on the purpose.

In the above-mentioned embodiments, observation of the cells with transmitted light is intended and the micro vessel is formed with polycarbonate that is easy to process. However, the present invention is not limited to polycarbonate. Any material that has excellent resistance to chemicals like the polycarbonate and does not cause chemical reactions with the cells can be used. For example, glass or acrylic resin can be used to form the micro vessel. When observation with transmitted light is not intended, the micro vessel can be made of an opaque material.

In the above-mentioned embodiments, a plurality of the cell immobilizing units is provided for a single micro vessel. However, only one cell immobilizing unit can be provided in each micro vessel. This enables observation of expression of the effect for a single cell can be performed with reliability.

In the first embodiment, the sucking unit is configured with a material having adhesiveness to the openings. However, adhesion is not always necessary. For example, mechanical means, such as screws can be bonded to the openings.

Further, in the first embodiment, a plurality of the openings provided in one micro vessel is operated by an outside sucking unit such as syringes. However, the present invention is not limited to these and, for example, depressions can be provided on the back side of the plate that constitutes the micro vessel at sites corresponding to the respective openings. A plate material that includes a diaphragm made of, for example, Si and having a foot portion and a thin film portion, and a piezoelectric element fixed to the thin film portion can be laminated in each depression. Moreover, the piezoelectric element can be driven to bend the thin film portion of the diaphragm to generate a negative pressure.

When the particles, e.g., the cells, are observed with transmitted light, this plate must be made of a transparent material such as an acrylic resin.

In the first embodiment, one sucking unit is provided for one opening. However, it can be configured to provide one sucking unit for all the openings and apply a negative pressure collectively.

However, care must be taken when the number of the openings is large because in that case the negative pressure in each opening may fluctuate.

In the above-embodiments, liquid is fed to the particle holding device to remove the free cells. However, the present invention is not limited to this configuration. For example, the suspension 13 in the micro vessel can be sucked and removed by using the removing device 50 as shown in FIG. 7.

The present invention can be applied typically to introduction of a gene or a drug solution into cells in the field of regeneration therapy and genome-based drug discovery. However, the present invention can also be applied to introduction of a trace substance into micro particles in the fields other than the fields of the regeneration therapy and genome-based drug discovery.

Although the invention has been described with respect to a specific embodiment for a complete and clear disclosure, the appended claims are not to be thus limited but are to be construed as embodying all modifications and alternative constructions that may occur to one skilled in the art that fairly fall within the basic teaching herein set forth.

What is claimed is:

1. An apparatus for producing particles having introduced therein a substance, comprising:
a feeding device that feeds a suspension containing a plurality of particles;
a holding device that receives the suspension from the feeding device, immobilizes at least one particles while the substance is being introduced in the particles and releases the particle once the substance is introduced in the particles;
an injecting device that injects the substance into the particles while the particles are immobilized by the holding device; and
a removing device that removes particles that are not immobilized by the holding device.

2. The apparatus according to claim 1, wherein the holding device comprises:
a vessel having a bottom and at least one opening in the bottom; and
a sucking device communicating to the opening and sucks the suspension containing the particles to thereby trap and immobilize the particle at the opening.

3. The apparatus according to claim 1, wherein the holding device includes a vessel having a bottom and an adhesive coating on at least a part of the bottom to adhere and thereby immobilize the particle.

4. The apparatus according to claim 1, wherein the feeding device comprises:
a suspension feed device that feeds the suspension containing the particles to the holding device;
a suspension feed controlling device that controls an amount of the suspension fed by the suspension feed device to the holding device; and
an agitator that agitates the suspension while the suspension feed device feeds the suspension to the holding device.

5. The apparatus according to claim 1, wherein the removing device comprises:
a liquid feed device that feeds a liquid to the holding device; and
a liquid feed controlling device that controls an amount of the liquid fed by the liquid feed device to the holding device.

6. The apparatus according to claim 1, further comprising:
a transporting device that transports the particles in which the substance has been introduced by the injecting device.

7. The apparatus according to claim 2, further comprising a transporting device that includes
a conveyor capable of conveying an empty vessel to a position where the suspension can be received in the vessel from the feeding device and to a position where the vessel with the particle into which the substance has been introduced is to be stored; and
a drive controlling device that controls driving of the conveyor.

8. The apparatus according to claim 2, further comprising a transporting device that includes
a working arm capable of grabbing a vessel and moving an empty vessel to a position where the suspension can be received in the vessel from the feeding device and to a position where the vessel with the particle into which the substance has been introduced is to be stored; and
a controlling device that controls driving of the working arm.

9. The apparatus according to claim 7, further comprising:
a storing device that stores and manages the particle into which the substance has been introduced.

10. The apparatus according to claim 8, further comprising:
a storing device that stores and manages the particle into which the substance has been introduced.

11. A method of producing particles having introduced therein a substance, comprising:
feeding a suspension containing a plurality of particles;
immobilizing at least one of the particles;
injecting the substance into the particle that has been immobilized;
releasing the particle that has been immobilized and into which the substance has been injected; and
removing particles that are not immobilized at the immobilizing.

12. The method according to claim 11, wherein the feeding includes feeding the suspension to a vessel having a bottom and at least one opening in the bottom, and the immobilizing includes sucking through the opening to trap the particle in the opening and thereby immobilizing the particle.

13. The method according to claim 11, wherein the feeding includes feeding the suspension to a vessel having a bottom and an adhesive coating on at least a part of the bottom, and the immobilizing includes causing the particle to adhere to the adhesive coating to thereby immobilize the particle.

14. The method according to claim 11, further comprising:
agitating the suspension before feeding the suspension, and
the feeding includes feeding the suspension in a controlled amount.

15. The method according to claim 11, wherein the removing includes feeding a liquid so that the particles that are not immobilized are transported by overflowing.

16. The method according to claim 12, further comprising:
transporting the particles in which the substance has been introduced at the injecting.

17. The method according to claim 16, further comprising:
storing and managing the particles that are transported at the transporting.
